(19) United States
(12) Patent Application Publication
(10) Pub. No.: US 2009/0069872 A1
(43) Pub. Date: Mar. 12, 2009

(54) Device and method for biological tissue stimulation by high intensity laser therapy

(75) Inventors: Damiano Fortuna, Firenze (IT); Leonardo Masotti, Firenze (IT)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: EL. EN. S.P.A., Firenze (IT)

(21) Appl. No.: 12/283,612

(22) Filed: Sep. 12, 2008

Related U.S. Application Data
(63) Continuation of application No. 11/668,970, filed on Jan. 30, 2007.
Application No. 11/668,970 is a continuation-in-part of application No. 10/417,672, filed on Apr. 17, 2003, now abandoned.
Application No. 10/417,672 is a continuation-in-part of application No. 09/885,616, filed on Jun. 20, 2001, now abandoned.
Application No. 09/885,616 is a continuation-in-part of application No. 09/325,165, filed on Jun. 3, 1999, now Pat. No. 6,527,797.
Application No. 09/325,165 is a continuation-in-part of application No. 08/798,515, filed on Feb. 10, 1997, now abandoned.

(51) Int. Cl. A61N 5/067

(52) U.S. Cl. 607/89

(57) ABSTRACT

A method for laser anti-inflammatory treatment of painful symptomatologies and for tissue regeneration includes generating a pulsed laser beam with laser at a wavelength between 0.75 and 2.5 micrometers. The laser energy is conveyed to a hand unit where the laser beam is preferably defocused. The operator then applies the defocused laser beam the skin of a patient in need of treatment. The average power density per pulse of the defocused laser beam on the skin being 8 W/cm² per pulse or more.

### RELATED APPLICATIONS

This application is a continuation of U.S. application Ser. No. 11/668,970, filed Jan. 30, 2007; which is a continuation-in-part of U.S. application Ser. No. 10/417,672, filed Apr. 17, 2003, now abandoned; which is a continuation-in-part of U.S. application Ser. No. 09/885,616, filed Jun. 20, 2001, now abandoned; which is a continuation-in-part of U.S. application Ser. No. 09/325,165, filed Jun. 3, 1999, now U.S. Pat. No. 6,527,797; which is a continuation-in-part of U.S. application Ser. No. 08/798,515, filed Feb. 10, 1997, now abandoned.

The entire teachings of the above applications are incorporated herein by reference.

### FIELD OF THE INVENTION

This invention relates to an apparatus and a method for therapeutic local treatment of living biological tissue by laser irradiation, and more particularly, to a noninvasive, nontraumatic method for stimulating living tissue.

The laser radiation is an electromagnetic wave characterized by a frequency ν, or, correspondingly, by a wavelength λ. Considering a sinusoidal waveform, the frequency ν is defined as the number of periods (i.e. of complete oscillations) per second. The wavelength λ represents the distance covered by the wave in a period. The two quantities are related to each other as follows: v=c/λ wherein c represents the speed of light (c=3×10⁸ m/s).

For a fixed wavelength such as that of a laser source, the effect of the laser on a tissue can be controlled by the following parameters:

POWER=energy per time unit measured in Watt (W).

INTENSITY=power per surface unit, or power density, measured in W/cm².

A further feature of a laser beam is the spot size, i.e. the cross-section size (measured in cm²) of the laser beam striking the tissue. The power and diameter of the spot are related via the intensity which, according to its definition, becomes smaller with the increasing diameter of the spot.

The emission of a laser may be either continuous or pulsed. Without considering the substantial differences between the numerous types of pulsed emission, the features of the waveform in the present case shall be pointed out.

Indicating by τ the length duration of the laser pulse and by T the interval of time between two successive pulses, the inverse of this interval is the frequency of pulse emission f=1/T measured in Hertz (Hz). The quantity D, given by the ratio between the pulse duration τ and the period T (D=τ/T) is referred to as duty cycle and varies from 0 to 1 (that is, between 0 and 100%).

By indicating the peak power of the pulse as Pp and Pm as the average or mean power per pulse, the following equation applies:

Pm=Pp×D

It is important to distinguish between the frequency v of the laser emission and the frequency f of pulses emission: both are measured in Hz, but are actually two different quantities, fully unrelated to each other. A laser radiation may have, at a given frequency v, either a continuous (f=0) or pulsed (f≠0) emission. Moreover, with f being equal it is possible to change the length of the pulse T or, correspondingly, the duty cycle D.

The object of the present invention is to provide an apparatus and a method for treatment by means of pulsed laser emission (f≠0) having high values of peak intensity (Pp/spot size) and of average intensity (Pm/spot size).

### BACKGROUND OF THE INVENTION

Since their discovery lasers have been advocated as alternatives to conventional clinical methods for a wide range of medical applications. For many years high-powered and highly focused lasers have been used to cut and destroy tissue in many surgical techniques. More recently, therapeutic and biostimulating properties of laser radiation were discovered. It is believed that laser radiation stimulates several metabolic processes, including cell division, synthesis of hemoglobin, collagen and other proteins, leukocyte activity, production of macrophage cells and wound healing. In this case the laser radiation acts as a stimulating agent on the cell activity and activates therewith the body's own healing capability.

Laser therapy is often used to give relief for both acute and chronic pain, resolve inflammation, increase the speed, quality and tensile strength of tissue repair, resolve infection and improve the function of damaged neurological tissue. This therapy is based on the application of narrow spectral width light over injuries or lesions to stimulate healing within those tissues.

The treatment with laser beams is painless and causes neither a macrochemical change nor a damage in the tissue.

Up to now the actual mechanism underlying the laser effects has not yet fully understood. According to one theory, the energy of laser radiation is incorporated in natural processes in a manner similar to that by which the “quanta” of light are incorporated in the chain of reactions of plant photosynthesis. Another theory is based on the assumption that cells and tissues have a certain reserve of free charge and are surrounded by a particular biological field such that the interconnections among organism, organs, apparatus and tissues are not determined by mechanisms of humoral, nervous and chemical regulations only, but also by more complex energetic connections.

The lack of understanding of the basic mechanisms underlying the effects of laser application resulted in a proliferation of several therapeutic devices and protocols using laser in very different ways and with different wavelength. Several U.S. patents have been granted for different apparatus and methods based on the laser application for therapeutic treatment of living tissue by laser irradiation. Among them the following are particularly relevant: U.S. Pat. No. 4,671,258 to Walker, U.S. Pat. No. 4,930,504 to Diamantopoulos et al., U.S. Pat. No. 4,931,053 to L'Esperance Jr., U.S. Pat. Nos. 5,445,146 and 5,951,596 to Bellinger, U.S. Pat. No. 5,755,752 to Segal.

The patent to Walker relates to a noninvasive and nontraumatic method of treating nerve damages in a human being, wherein essentially red light is used. In the preferred embodiment disclosed therein a HeNe laser is used with a wavelength of approximately 632.5 nm.

The patent to Diamantopoulos et al. discloses a device and method for laser treatment of living tissues, wherein an array of monochromatic radiation sources emitting at different wavelengths is used. Preferably at least three different wavelengths are used. The radiation sources are arranged within the array such that radiation of at least two different wavelengths pass directly or indirectly through a single point located within the treated tissue. According to Diamantopoulos et al. only the matched action of several wavelengths can produce the desired therapeutic and biostimulating effect. Radiation sources operating both in continuous or in pulsed mode are disclosed but continuous mode is preferred. If a continuous source is used the laser power is generally in the range of 5 mW to 500 mW. If a pulsed laser source is employed the peak power can reach 70 W, but the average power is kept below 100 mW.

The patent to L'Esperance discloses the use of at least two laser beams in the visible red or low infrared for enhancing or promoting vascular or other tissue growth in a living body tissue. As in Diamantopoulos also in the patent to L'Esperance the therapeutic effect is only given by the matched action of two laser. This method uses a power density in the order of micro W/cm².

Bellinger and Segal use lasers, both in continuous or pulsed mode, to cause the amount of optical energy absorbed and converted to heat in the tissue to be a within a range bounded by a minimum absorption rate sufficient to elevate the average temperature of the irradiate tissue to a level above the basal body temperature, but which is less than the absorption rate at which tissue is converted into a “collagenous substance.” According to this method a therapeutic warming effect is produced within the irradiate tissue. In both cases the preferred wavelength is 1,064 nm. Bellinger teaches to use a Nd:YAG laser, whereas Segal discloses the use of a In:GaAs diode laser. The power output level is always of less than 1,000 mW. Bellinger discloses a protocol of therapy in pulsed mode using an energy density in the range of 0.1 J/cm² to 15 J/cm² and both pulse-on time and pulse-off time are in the range of 0.1 to 9.9 seconds (in other words the frequency range is from 0.05 Hz to 5 Hz).

It is interesting to note that all the above mentioned patents, as well as most works in this field, refer to use laser at “low” or “medium” power level. This kind of therapy is now popularly referred to LLLT (Low Level Laser Therapy) or LILT (Low Intensity Laser Therapy). The power range used in LLLT is between few mW and 1,000 mW at most.

LLLT has become a popular treatment in a variety of medical disciplines. This therapy is used with some success but results are obtained only slowly and are inconstant. The degree of therapeutic effect achieved is variable and heavily depends upon the dosage of luminous wave and to the exposure rhythm. Applications of several minutes are repeated at intervals of several days and often repeated for months.

### SUMMARY OF THE INVENTION

In view of the unsatisfactory results obtained with LLLT, the object of the present invention is to provide a more efficient device and a better method of laser treatment with which faster and more constantly reproducible results can be obtained.

A further object of the present invention is to provide a device and a method for the treatment of chronic degenerative pathologies, such as osteoarthritis pathologies characterized by damage to bone and cartilage tissues.

The invention is based on the recognition that the stimulating effect is not due exclusively to the specific wavelength of the laser adopted, but also to the intensity, i.e. the power density. For obtaining this, laser pulses must be used which are much higher and quicker than those currently used in LLLT therapy.

Accordingly, the present invention relates to a protocol of therapy, and a relevant device for “high” intensity radiation for biostimulation of living tissue without exposing it to damaging thermal effects, thanks to a special defocusing system. This treatment reduces pain, inflammation and oedematous component, enhances the healing of tissue, stimulates microcirculation, cell division, DNA production, decreases muscle spasm and increases cellular ATP levels.

One important aspect underlying the method of the present invention is that two distinct minimum thresholds have been found for the power density of the laser radiation: a first threshold above which mainly an anti-inflammatory effect becomes apparent and a second higher threshold above which the therapeutic effect of cellular proliferation induced by laser stimulation becomes appreciable. In vivo experiments on a human degenerative joint pathology model allowed to understand the action mechanism according to which laser radiation leads to the abovementioned cellular proliferation effect. For the first time an explanation of the reason why laser radiation, and in particular Nd:YAG laser has a biostimulating and regenerative effect has been given. This explanation is consistent with all the clinical results reported in the literature, both the positive as well as the negative ones. This theory goes well beyond a simple list of effects at cellular level obtained by means of laser energy application, effects which up to now have been obtained mainly by in vitro experiments rather than by in vivo applications.

Essentially, according to a first aspect the present invention is based on the recognition of the fact that by applying an appropriately defocused laser beam, having specific characteristics in particular in terms of power density, at a given area of the epidermis of a patient afflicted by painful symptomatologies of various origins (deriving, for example, from past and recent traumas, arthritis, arthrosis or rheumatism), the stimulation of the nerve ending by means of the incident energy causes a gradual reduction, and in the end the disappearance, of the pain and a quick recovery of articular mobility and functional aspects.

According to a second aspect of the present invention, if suitable power density levels are achieved an actual proliferative explosion at cellular level has been surprisingly obtained, which in the specific case of degenerative joint diseases leads to a chondrogenic action, i.e. to a reconstruction of cartilage tissue, which up to now has not yet been reached with any drug-based therapy.

Therefore, according to the present invention, two distinct kinds of actions for the treatment of two different kinds of pathologies are obtained. In the case of acute painful affections in which pain is dominant, a protocol of therapy is applied which is characterized by a mean or average power intensity per pulse on the skin surface equal to or higher than 8 W/cm² and preferably comprised between 8 and 30 W/cm². This range is higher than the minimum power density threshold which shall be passed in order to obtain an anti-inflammatory effect with a Nd:YAG laser.

If chronic-degenerative pathologies must be treated, an average power intensity per pulse within a range of between 30 and 70 W/cm² per pulse is used. Higher power densities might lead to tissue damages and reduce the regenerative effect and are therefore avoided. This range is higher than the minimum power density threshold necessary to obtain a therapeutic-regenerative effect by means of a Nd:YAG laser.

Macroscopic and histological control have shown that:
both power densities have an anti-inflammatory effect;
the lower power density level has a minimum chondrogenic effect which consists in the appearance of a limited number of young isogenic group;
application of the higher power density level resulted in a real explosive proliferation with an apparent hyperplasia and hypertrophy of the young isogenic groups. A strong cellular proliferation is therefore obtained at the higher power density level, revealed by the presence of extended neochondrification areas.

It is important to note that in spite of the high power density used, the temperature increase at skin level must be kept to a minimum since too high a temperature increase would result in tissue damages as well as in an inhibiting effect on the biostimulating-regenerative mechanism. In order to achieve this result according to the invention a pulsed laser is used.

In general terms, the interaction of an electromagnetic radiation with a biological tissue depends upon the radiation wavelength and upon the optical properties of the tissue. A laser beam directed orthogonal to the surface of the tissue is partly reflected back due to the variation of impedance index when passing from the surrounding ambient (air) and the tissue. The remaining fraction of the laser beam energy is transmitted to and through the tissue and is absorbed and diffused several times by different chemical substances contained in the tissue.

The purpose of the invention is to select emission parameters such that the penetration depth of the laser beam is improved in order to reach locations arranged deeply within the body of a patient under treatment, without damaging the tissues which are passed by the laser beam or the tissues surrounding the volume subject to the laser treatment. A deep penetration of the laser radiation allows laser treatment of lesions e.g. of the cartilage tissue located at a relatively deep position within the body without damaging the surrounding biological tissue.

According to the literature, the degree of penetration of the laser energy through the biological tissues depends on the coefficient of tissue absorption and on the fluence (energy per surface unit: J/cm²) of the laser beam, i.e. the density of the beam energy. The energy per surface unit is given by the power density multiplied by the time of irradiation. Therefore the degree of penetration of the laser beam into a biological tissue directly depends upon the wavelength of the laser beam and upon the power of the laser beam: the higher the power of the beam the higher the penetration depth into the tissue under treatment.

Details on the effect of these parameters on the penetration depth of a laser beam in biological tissues are discussed in K. Doerschel et al, “Photoablation”, SPIE, Vol. 1525 Future Trends in Biomedical Applications of Laser (1991), p. 253-278. Dependency of the penetration depth on the above mentioned parameters is shown in FIG. 9 on page 261 of Doerschel et al. The higher the absorption coefficient the poorer the penetration of the radiation through the tissue. Table 2 (page 264) of Doerschel show the dependency of the absorption coefficient upon the wavelength of the incident laser radiation. The data reported by Doerschel et al show that a CO₂ laser (wavelength 10,600 nm) has an absorption coefficient of 600 cm⁻¹ and has therefore a very low penetration capability, while a Nd:YAG laser (wavelength 1,064 nm) has a very low absorption coefficient (4 cm⁻¹) and a much better tissue penetration capability.

Additional information on the penetration depth of different laser sources is presented in J. Tuner et al, Laser Therapy. Clinical Practice and Scientific Background, Prima Books, 2002, pages 40-43.

As stated above, in order to reach—with an intensity higher than the activation threshold—tissues which are deeply under the skin of the patient under treatment, high power values have to be adopted, at the same avoiding tissue damages due to photothermal phenomena.

In continuously emitting laser systems, an increase in the emission power results in an increased emitted energy, which is the integral of the power in time. Part of said energy is transformed into heat in the irradiated tissues. The speed of propagation of the heat in water (the biological tissues being mainly formed by water) is much lower than the speed of propagation of the electromagnetic radiation in the tissue. This has as a consequence that the heat generated by the laser energy in the tissues accumulates at a certain depth under the skin of the patient being treated with consequent negative effects due to temperature increase.

The diffusion length of the heat in a biological tissue is an important parameter for controlling the thermal effects during laser treatment. Such length L is given by

L²=4Kt

where K is called thermal diffusivity of the material where the heat is propagated, and is a function of the thermal conductivity, specific heat and density of the material; t is time.

From the above formula, given that for water K=1.43 10⁻³, heat energy propagates in water at 0.8 mm per second. By putting the diffusion length L equal to the penetration depth of a laser radiation, the relaxation time is obtained as follows

tᵣₑₗₐₓ=¼Kx²

where tᵣₑₗₐₓ is the relaxation time, K is the thermal diffusion coefficient of the tissue and x is the penetration depth. For a Nd:YAG laser, being the penetration depth equal to ¼ cm, and assuming for K the value 0.00143 (the value of water) the relaxation time is 312.5 seconds. This means that if a Nd:YAG laser is used to reach deep penetration into the tissue, a rather high thermal relaxation time is obtained. This causes a slow temperature increase in the tissue under treatment and a slow thermal dissipation. Such a slow dissipation might lead to heat accumulation and consequent damages in the tissues under treatment.

In order to avoid thermal accumulation and excessive temperature increase in the tissue under treatment, it is necessary to provide sufficient time between successive laser pulses, for the heat to dissipate. To achieve an activation threshold, i.e. a sufficient amount of energy for obtaining the desired therapeutic effect, on the other hand, this requires the use of high peak power values.

An additional important parameter having an influence on thermal accumulation and therefore on the temperature increase is the overall volume of tissue under treatment. Keeping the irradiated surface (i.e. the laser spot) and the irradiated energy constant, an increase of the peak power per pulse increases the irradiated volume. The reason for this is that a higher peak power provokes a deeper penetration of the laser in the tissue, and therefore an increase in the overall volume absorbing the laser energy. The penetration depth is understood as the depth at which the density level of the laser radiation is higher than the activation threshold.

On the other hand, the same amount of irradiated energy causes a temperature increase which is inversely proportional to the irradiated volume: the larger the irradiated volume the smaller the temperature increase. Therefore, and contrary to what might appear at first glance, an increase of the peak power of each laser pulse improves the conditions of treatment from the point of view of tissue temperature control.

It has been therefore recognised that in order to obtain an effective treatment of the deep tissues without damaging more superficial and surrounding tissues, a pulsed laser source with low pulse frequency and short pulses (i.e. low duty cycle values: short T on times and long T off times) has to be used, in combination to high peak power values per pulse.

The area of the laser spot is also of some importance, because the larger the diameter of the spot, the lower the scattering angle. This results in a deeper penetration, more uniform diffusion of the radiation in the tissue, and therefore an increased therapeutic effect. By proper selecting the above discussed parameters, the tissue temperature in the treated volume is kept below 45° C. or even lower, and preferably below 40° C. If required, cooling of the skin of the patient under treatment can be additionally provided.

Proper control of the heat accumulation and avoidance of thermal damages is achieved by:
selection of proper laser wavelength, e.g. and in particular Nd:YAG lasers having a low absorption coefficient;
use of pulsed laser sources;
high peak power values per pulse;
low frequency of the pulses, i.e. long time between two subsequent pulses;
low duty cycle, i.e. short emission time per pulse (Ton);
large laser spot.

High peak power values (kWatt/cm²) additionally allows a further effect to be exploited for therapeutic purposes, namely the photomechanical effect. This effect substantially consists in a sort of massage of the tissue subject to irradiation, when the peak power of the pulse, the pulse duty cycle and the pulse frequency are properly selected.

The photomechanical effect adds to the photochemical effect of the laser irradiation.

In view of the above, HILT (High intensity laser therapy) distinguishes over Low Level Laser Therapy (LLLT) in respect of the purposes to be achieved and selection of operating conditions and parameters to achieve said purposes and objectives. As far as the purposes are concerned, the main object of the HILT is non-painful and non-invasive therapeutic treatment of deep lesions, such as lesions of the articular cartilage. Secondary objectives of the HILT are:
achievement of photomechanical effects in addition and in combination to photochemical effects, thanks to the high peak power values adopted;
transfer of high-energy photons at the deepest level possible within the tissues;
control of tissue temperature below 45° C. and preferably below 40° C.

The above objectives are achieved by following some general rules:
the deeper the penetration of the laser radiation, the longer the time between subsequent laser pulses, to allow for thermal dissipation;
the higher the energy content per laser pulse, the lower the pulse frequency, i.e. the frequency at which the laser pulses are repeated;
the higher the power level per pulse, the lower the fluence;
the higher the peak power of each pulse, the shorter shall be the pulse duration (low duty cycle);
the higher the peak power, at constant spot area, the larger will be the volume interested by the radiation and therefore the lower will be the increase in temperature due to heat accumulation;
the higher the energy per pulse, the shorter will be the total exposure time to the laser radiation;
the shorter the total exposure time to the laser radiation, the lower will be the heat accumulation.

It has been observed that the frequency of the laser pulses should preferably be between 1 and 40 Hz. Such low value of the pulse frequency allows optimal thermal dissipation. The t-on time, i.e. the duration of each pulse is preferably between 1 and 300 microseconds and the energy per pulse is between 0.03 and 1 Joule. Heat removal through the skin of the patient under treatment can be added as a means to limit or control tissue temperature.

The laser beam is de-focused to generate a spot of substantially circular form, with a diameter of between 4 and 10 mm and preferably between 5 and 7 mm.

The laser emission wavelength is preferably between 0.75 and 2.5 micrometers and preferably in the range of 1,064 nm. Different wavelengths can be adopted, which are characterised by a low absorption coefficient, preferably with an absorption coefficient equal to or lower than 50 cm⁻¹ and more preferably equal to or lower than 15 cm⁻¹ in normal soft biological tissue. In addition, the wavelength chosen should not correspond to peak absorption wavelengths of typical tissue substances, such as melanin, hemoglobin or other chromophores.

It will be clear from the above that, especially when high peak power levels are used, such as for the treatment of chronic degenerative pathologies, strict control of the treatment conditions are important. The peak power should be as high as possible compatibly with the need to avoid thermal damage of the tissues. The actual operating conditions strongly depend upon the phototype of the patient under treatment. According to a further aspect of the present invention, the skin temperature can advantageously be detected in a continuous or discontinuous manner, such that the actual skin temperature is kept under control. The irradiation conditions are set such as to have the most effective irradiation (i.e. the deepest penetration and the highest power levels), without nevertheless exceeding threshold temperature values, e.g. 40° C. or 45° C. of the skin temperature. This can be achieved by a temperature sensor arranged on a handpiece. A photodetector for determining the phototype of the patient under treatment could also be combined to the handpiece. In addition to provide proper control during treatment, the temperature sensor and photodetector are useful in order to determine the quantity of energy which is absorbed by the tissue and transformed into heat or else reflected by the skin. Knowing the total energy emitted by the source and impacting the skin, the value of the energy actually reaching the deeply located tissues to be treated can be determined with sufficient precision.

According to a different embodiment of the invention, frequency of the laser pulses may be selected between 5 and 100 Hz, preferably between 5 and 50 Hz, and even more preferably between 10 and 40 Hz, while optimum results can be achieved with frequencies between 15 and 25 Hz.

According to a further advantageous feature of the invention, a pulse duration T can be used which can vary between 100 and 500 microseconds, and preferably between 100 and 300 microseconds and even more preferably between 200 and 300 microseconds. This avoids accumulation of thermal energy in the tissue. The thermal energy impacting on the tissue during one pulse is dissipated before the next pulse arrives. Temperature control of the tissue is thus obtained.

Moreover the present invention also relates to a device for laser therapy comprising a first laser source which produces a single therapeutic laser radiation, a first conveying means for conveying the laser energy to a hand unit, and optical defocusing means for defocusing the laser beam, which are positioned in the path of the laser beam.

According to a preferred embodiment, the conveying means is formed by an optical fiber, in front of the output end of which the optical defocusing are arranged.

It has been observed that particularly strong therapeutic effects, and therefore rapid results in the reduction of painful symptomatology as well as in the stimulation of tissue regeneration, are obtained by using a pulsed laser source which emits at a wavelength between 750 nanometers and 2.5 micrometers and preferably between 900 nanometers and 1.2 micrometers, and with an energy level between 30 and 500 mJ per pulse, preferably between 30 and 300 mJ per pulse and more preferably between 10 and 200 mJ per pulse. A particularly suitable laser source is the Nd:YAG laser with a wavelength of 1.064 micrometers. The frequency of the pulses as well as their duration are also parameters which have a considerable influence on the effectiveness of the treatment. These values clearly distinguish the present invention over the LLLT therapy of the prior art. In particular the mean pulse intensity is up to 200 times higher than the one used in LLT therapy.

It should also be appreciated that the peak power is in the order of some kW and the average power is bigger than 1 Watt. These values are clearly greater than those used in the prior art therapeutic methods, in particular those disclosed in the previously cited U.S. Patents. On the other hand the pulse duration is extremely shorter. In this way the minimum stimulation threshold can be passed and the desired result achieved.

The diameter of the laser spot can be between 4 and 10 mm and preferably between 5 and 7 mm. Contrary to that, the prior art methods require focusing means in order to achieve the desired power density with the low power levels suggested therein.

The hand unit, where the laser optical path ends and the defocusing means are arranged, can be held by the operator at the appropriate distance from the epidermis of the patient undergoing treatment. In order to make use safer and easier for the operator, however, the end unit is in a preferred embodiment provided with a distance element to hold said optical means of defocusing at the predetermined distance from the body of a patient to whom the treatment is being applied, avoiding the necessity of determining and manually maintaining the optimum distance.

Again for the purpose of facilitating use of the device, it can be provided with a second laser source which emits at a wavelength in the visible range, and optical fiber or equivalent means for conveying the laser beam generated by said second source towards the hand unit. This second laser source is only a marker and it has no therapeutic properties.

According to an embodiment of the invention, the pulsed laser beam has a duty cycle between 0.01 and 1% and preferably between 0.01 and 0.1%. The duty cycle indicates the ratio between Ton and T=Ton+Toff in a laser pulse, where T=Ton+Toff is the total duration of a pulse cycle, Ton is the time interval during which the laser beam is on and Toff is the time interval during which the laser beam is off. The shorter the Ton time interval, the lower the duty cycle. A low duty cycle in combination with a high mean power value results in very high peak power values per pulse. Low duty cycles allow sufficient time between subsequent Ton periods during which heat can be removed from the treated tissue, avoiding tissue damages, in spite of the extremely high peak power values achieved during each Ton period.

In a preferred embodiment the mean power per pulse is between 5 and 100 W and preferably between 6 and 70 W or even more preferably between 6 and 60 W. In an embodiment of the invention the pulse frequency is lower than 100 Hz. An advantageous pulse frequency range is between 0.1 and 60 Hz and preferably between 0.5 and 40 Hz. Ton intervals lower than 300 microseconds and preferably between 1 and 250 microseconds are particularly suitable. According to an embodiment of the invention, pulse durations (Ton times) are used ranging between 5 and 250 microseconds or preferably between 50 and 220 microseconds or even more preferably between 70 and 200 microseconds.

According to an embodiment of the invention, the pulsed laser beam achieves peak powers higher than 500 W. In an embodiment of the invention, peak powers up to 60,000 W per pulse or more are possible. Particularly useful results in terms of tissue regeneration are achieved by peak power values between 3,000 and 60,000 W, with a frequency of the pulsed laser between 0.3 and 70 Hz, preferably between 0.5 and 40 Hz. and pulse durations (Ton times) between 50 and 250 and preferably 70 and 200 microseconds. In an embodiment of the invention, pulse durations lower than 150 microseconds and between 100 and 130 microseconds are used.

According to an embodiment the laser spot is chosen such that peak power per surface unit higher than 2000 W/cm² and preferably higher than 2500 W/cm² are applied. According to one aspect of the invention, peak power values per square unit between 3000 and 30,000 W/cm² are suitable, values ranging between 13,000 and 30,000 W/cm² being particularly preferred.

According to an embodiment of the invention, high power-pulsed laser beams are generated by a solid state laser source, i.e. a laser source formed by a doped mono-crystal structure. A suitable solid state laser source is a Nd:YAG laser. This laser can emit a sufficiently high-power pulsed laser and has an emission wavelength of 1.064 nm, a particularly advantageous wavelength because said radiation can be transmitted through biological tissues of interest in the present application and achieve in-depth cartilage structures on which tissue regeneration is required.

When a pulsed laser beam impacts a medium, an elastic pressure wave is generated in the medium. The intensity of the waves is directly proportional to the intensity of the laser beam and inversely proportional to the pulse duration time. It also depends on the properties of the light and on the physical-chemical structure of the medium.

According to an embodiment of the invention, tissue regeneration is enhanced by exploiting said photomechanical effect induced by the high powered-pulsed laser beam on the tissue being treated, in combination with a direct photochemical effect induced by the laser photons on the cells. Cartilage tissue is characterized by an extra-cellular matrix, wherein the tissue cells are contained. The photomechanical effect induced by the pulsed high intensity laser causes repeated contraction and expansion of the extra-cellular matrix and of the cells contained therein. This mechanical effect stimulates a chondrogenic action. The direct photochemical effect, i.e. direct absorption of energy from the laser photons by the cellular structure, controls the cell differentiation such that healthy hyaline cartilage tissue is regenerated rather than fibrous cartilage tissue.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

FIG. 1 shows the hand unit of the device and, diagrammatically, the laser sources and the control systems.

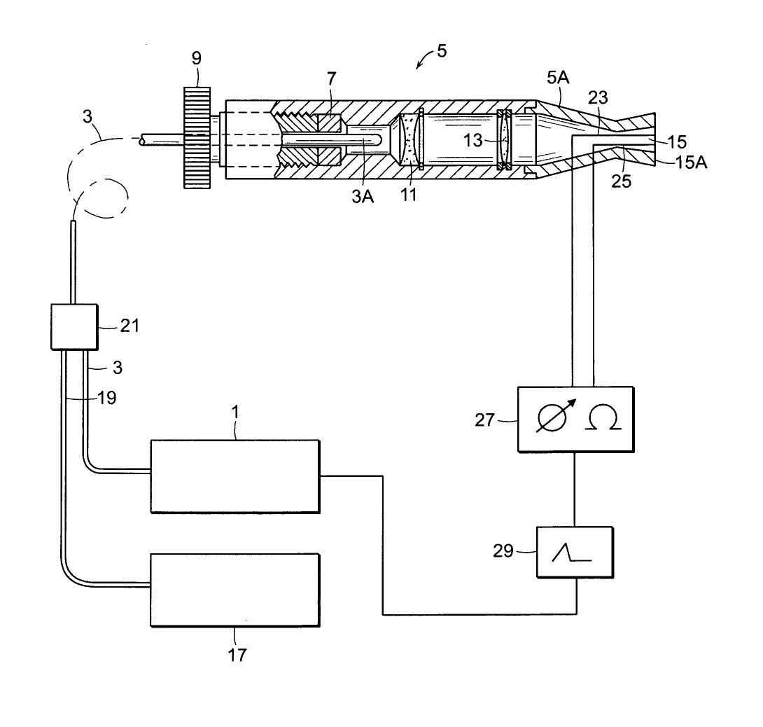

### DESCRIPTION OF THE PREFERRED EMBODIMENTS

In the attached diagrammatic drawing, 1 indicates a laser source, preferably a Nd:YAG laser with emission at 1.064 micrometers, connected by means of an optical fiber 3 to a hand unit 5. Inside the hand unit, the output end 3A of the optical fiber 3 is fixed by means of an elastic sleeve 7 and clamping nut 9. Arranged facing the end 3A of the optical fiber 3 is a defocusing optic 11, 13.

The hand unit 5 ends in a converging end 5A to which is fixed a distance piece 15 with a surface 15A which is brought into contact with the epidermis E of the patient to whom the treatment is being applied. In this way, the defocusing optic 11, 13 is always held at a predetermined distance from the epidermis. In this way, once fixed, the energy is determined only by the energy density.

A second laser source 17 which emits continuously at a wavelength in the visible range introduces a laser beam into the fiber 3 by means of an auxiliary optical fiber 19, a connector 21 and a mixer. As an alternative and equivalent, the second laser source can send the laser beam into a known device for coaxial mixing of the two laser beams. The two beams made coaxial are then sent to a known device for introduction into the fiber.

In this manner, the treatment zone is illuminated and can be seen by the operator in the presence of the distance piece 15 also if this is open or made of a transparent material.

Associated with the distance piece 15 are two electrodes 23, 25 connected to a resistance measuring device 27. This measures the resistance of the epidermis in the region of the zone of application of the hand unit 5 and, by means of a trigger signal generator 29, generates a control signal for the laser source 1 in such a manner that the latter emits pulses at the frequency and of the duration desired when the hand unit 5 is in the region of the trigger point, where the resistance measured by the measuring device 27 is low.

The features of the laser emission from source 1 can be as follows. During each period T of the pulsed laser emission a pulse of duration T is generated followed by an “off” interval. As stated above, the ratio D between the duration of the pulse and the period T is the duty cycle (D=τ/T) of the laser emission. The peak power is designated Pp, and is linked to the mean or average power per pulse Pm via the period T and the duty cycle D as indicated above.

The dimension of the spot generated by the laser beam on the skin of the patient being treated depends on the optical features of the defocusing means and on the distance between the optical defocusing means and the skin. The power density, i.e. the power per surface being a critical value, the dimension of the spot is an important parameter characterizing the method of treatment. This is selected such that the power density falls within the range indicated above, depending upon the particular application.

While the various parameters of the laser emission may vary within the above mentioned ranges, the following values can be indicated as a preferred example of the method for the treatment of painful syptomatologies:
Wavelength of the laser source: 1.064 micrometers (Nd:YAG laser source);
Pulse frequency (1/T): 25 Hz;
Pulse duration (τ): 200 microseconds;
Mean power density per pulse: 10 W/cm²;
Laser spot diameter: 5 mm;
Energy per pulse: approx. 312 mJ.

Similar values of the above listed parameters can be used for the stimulation of tissue regeneration but with an increased mean power density per pulse of 35 W/cm² and consequently an increased energy per pulse.

### The Action of Laser Radiation on the Tissue

The relationship between dose of laser radiation and efficiency of the treatment has always been considered important for the therapeutic action of the laser. This fact has been widely reported in the literature, based on in vitro experiments.

In vivo experiments conducted on knee joint in rats, have shown that a power density of 5.8 W/cm² is not sufficient to pass the activation threshold. (Usuba M, Akai M, Shirasaki Y: Effect of low level laser therapy (LLLT) on viscoelasticity of the contracted knee joint: comparison with whirlpool treatment in rats, Laser Surg Med 1998, vol. 22 pp. 81-5).

The present invention is based on the surprising recognition of the importance of the intensity of the laser radiation on the skin rather than the “dose” thereof, i.e. the total energy applied during the whole treatment.

### Mechanism of Action

In order to fully understand and describe the way of action of the laser radiation on an injured biological tissue, it is crucial to consider the clinical phenomena observed during the laser therapy. At least four different levels of investigation shall be considered: clinical, biochemical, molecular biology-related, and physical.

As a matter of fact, by putting physical considerations before the biochemical aspects, it is not possible, for example, to reconcile the clinical efficacy of the radiation at 10,600 nm (CO₂ laser) with its optical properties related to biological tissues. That being stated, it is therefore crucial to first consider the therapeutic effects of the laser, as reported in the literature of the last twenty years: i.e. the anti-inflammatory, biostimulating, antalgic, antiedemic and lipolytic effects.

In the animal model of osteoarthritis pathology it has been found that the application of the method according to the invention causes a drop of PCR (reactive protein-C) values. This is due to a reduction of the incretion of cytokines such as IL-6, IL-1, and TNFα. Incretion is a glandular secretion which is intended to remain and act inside its generating organism.

The cytokines reduction is not due so much to a direct effect of the laser action over these or other phlogogenic cytokines, as to the stimulation induced by the laser on certain grow factors, such as TGFβ and IGF-1, which have an antagonizing effect over said cytokines. Cytokines are proteinic, hormone-like factors produced by a wide range of cells. They exert a number of different biological effects, among which the control of the inflammatory, grow and cellular differentiation processes, as well as of the immunological responses processes of a host, by acting as intracellular messengers. The best known cytokines are the tumoral necrosis factor (TNF), the interferons and cytokines. Also known are cytokines of phlogogenous type which activate catabolic processes leading to tissues destruction, and anabolic cytokines which, on the contrary, promote the regenerative processes.

Accordingly, the laser radiation does not provide any blocking action on any cellular structure or product (for example, IL-1β, TNFα, IL-6), but can promote, with a readily available energy, the anabolic cytokines able to reverse the catabolic process under way.

This stimulation actually takes place by acting both on the cellular receptors, having an intrinsic tyrosinchinasic activity, and on those which utilize receptors associated to intracytoplasmic tyrosinchinase.

Belonging to the former type is a group of receptors having the insulin as prototype. In particular, the group includes the receptor for the insulin-like-growth factor (IGF-1), the receptor for the transforming grow factor P (TGFβ), the receptor for epidermic grow factor (EGF) and that for platelet-derived grow factor (PDGF). Following the activation by interaction between the receptor and the hormone, it is possible to modulate the activity of other molecules involved in the cellular proliferation.

In other words, these receptors have such a structure as to be able to directly change the cellular activity, once they have been activated by the specific hormone (e.g. IGF-1).

The receptors of the second group, which utilize intracytoplasmic tyrosinchinase, are also called receptors of “GH/cytokines,” since to this group belong the receptors of GH, prolactin, erythropoietin and of a number of cytokines.

The laser favors, in the first place, the tyrosinchinasic activity of the receptors having intrinsic activity (thus increasing the IGF-1, TGFβ, EGF, PDGF factors) and secondly those having intracytoplasmic tyrosinchinase (by improving the GH effect).

To understand the operating mechanism generated by the laser it is worth remembering how the enzymatic systems work. These operate in a way similar to the inorganic catalysts, but have a much higher specificity of action. In fact, the enzyme adsorbs selectively the sublayer on which it acts and becomes intimately joined therewith.

Once they have reacted, the molecules adsorbed by the enzyme are less strongly bonded and move away from the enzyme which becomes available again. It should be born in mind that the major object of an enzyme (similarly to a catalyst) is to reduce the triggering (kinetic) energy necessary for the molecules to enter a given reaction cycle. The catalyst and the enzyme, therefore, reduce the energy requirements, that is, the energy threshold the molecule has to get over to start the reaction.

Under stress conditions, such as those induced by chronic infections, an increase in the phlogogenous cytokines takes place, which brings about the activation of intracytoplasmic tyrosinchinase receptors with a competitive interference over the GH. This phenomenon could provide an explanation of the reason why the anabolic phenomena of the cell are not completely blocked, but are in fact prevented because of a phenomenon of enzymatic and energetic competition.

In this situation, the readily available energy from the laser favors the pathway of intrinsic tyrosinchinase receptors, not that of the intracytoplasmic ones (already engaged by the phlogogenous cytokines), with a preference for such grow factors as the (IGF-1), Transforming Grow Factor β (TGFβ), epidermic grow factor (EGF) and platelet-derived-grow factor (PDGF), which tip the homeostatic cellular scales in favor of the anabolic pathway instead of the catabolic one.

At this level, the laser operates in two distinct ways:
directly on chemical reagents: this is probably the first pathway of intervention; in fact, during a chronic and/or degenerative inflammation, a saturation of the cytoplasmic tyrosinchinase takes place due to the stimulation of the “GH/cytokines” induced by the phlogogenous cytokines (IL-1β, TNFα, IL-6 etc.) which largely prevail over the anabolic cytokines (GH, IGF-1, TGF, etc.). Under this condition, the availability of kinetic energy delivered by the laser radiation would favor the direct access of the cellular reagents to the cycle of metabolic reactions, induced by the anabolic cytokines, also in case of a shortage of tyrosinchinase enzyme (shortage due to the action of the phlogogenous cytokins). As a rule, with no enzyme action, it is not possible to activate the anabolic reactions, as the required energy is too high: the laser does provide for such energy. In this mode, the cell would have the possibility of starting again the anabolic activities interrupted by the inflammatory condition. The essential difference between the laser and the medicines having anti-inflammatory activity lies in the fact that the laser stimulates the anabolic cytokines towards resuming their metabolic efficacy and does not block any activity, contrary to anti-inflammatory drugs which inhibit some metabolic pathways (including those of the phlogogenous cytokines) without promoting anything. It is interesting to note how the blockage of the TNFα determines only a slowing down of the degenerative phenomenon under way, but not a reversal of tendency, contrary to what can be observed in vivo when using laser radiation. The absence of reversal of tendency, in spite of the blockage of TNFα, can be explained by considering that the other phlogogenous cytokines go on with their antagonist activity by binding the tyrosinchinase;
indirectly on the tyrosinchinase (membrane, cytoplasm): in this case the laser makes greater amounts of tyrosinchinase available by activating its enzymatic precursors. Such higher quantity of intracytoplasmic tyrosinchinase allows the occurrence of metabolic activities induced by the paracrine role of GH (anabolic cytokine).

In conclusion, the laser radiation at the power intensity levels disclosed above leads at first to an initial by-pass effect by promoting the metabolic activities of the grow factors. Afterwards, it makes greater quantities of intracytoplasmic tyrosinchinase available, which are useful to the pathway of GH.

It is known that the TGFβ has, at high doses, an antagonist effect versus the TNFα, the latter having a significant role in the genesis of osteoarthritis phenomenon. Also known is the fact that IL-1β and TNFα an increase the availability of receptors for glicocorticoids. All of these, in the case of inflammation cronicity, contribute to orienting the organism towards the catabolic pathway rather than the anabolic one, thereby increasing the degenerative phenomena. Lopez Calderon et al. (see Lopez Calderon A, Soto L., Martin A I. Chronic inflammation inhibits GH secretion and alters serum insulin-like-growth factor system. Life Science. 1999:65(20):2049-60) have reported the results of in vivo experiments describing that the chronic inflammation inhibits the secretion of GH and alters the serum levels of IGF-1.

A whole string of positive effects due to the axis GH-IGF-1 in the homeostatic scales of the organism is known, said axis being modified when a cachexic or degenerative phenomenon takes place.

The laser radiation, when delivered with an intensity sufficient to pass the activation threshold, is able to promote the cellular activities without inducing any “pharmacological blockage” of any type. It is known, in fact, that a significant limit of the anti-inflammatory drugs lies in the fact that, by acting with a blocking effect on some biological functions, they always cause undesired side effects (the TNFα, for example, induce a serious weakening of the immune system).

In short, the laser, by supplying readily available kinetic energy, favors in the first place the activation of the receptor pathway for intrinsic tyrosinchinasic activities, notwithstanding any enzymatic deficiency. This promotion triggers a series of intracellular and extracellular phenomena which affect, by improving them, the grow factors IGF-1, TGF, EGF, PDGF. In the second place the activation of the intracytoplasmic tyrosinchinase takes place, which boosts the effect of GH by restoring the axis GH/IGF-1, and of the cytokines.

This explains why, under particular conditions, the laser has no anti-inflammatory effect, but does have a prophlogistic effect which improves and sustains the immune system.

While this invention has been particularly shown and described with references to example embodiments thereof, it will be understood by those skilled in the art that various changes in form and details may be made therein without departing from the scope of the invention encompassed by the appended claims.

What is claimed is:

1. A method of laser treatment for stimulating regeneration of cartilage in a patient, comprising:
generating a pulsed laser beam having kilowatts of peak power; and
applying the pulsed laser beam to the cartilage of the patient.

2. The method of claim 1, wherein the patient is afflicted by a chronic degenerative pathology.

3. The method of claim 1, wherein the pulsed laser beam has a wavelength between 0.75 and 2.5 micrometers.

4. The method of claim 1, wherein the pulsed laser beam has a wavelength between 0.9 and 1.2 micrometers.

5. The method of claim 1, wherein the pulsed laser beam has a wavelength of 1.064 micrometers.

6. The method of claim 1, wherein the pulsed laser beam is generated by a Nd:YAG laser source.

7. The method of claim 1, further comprising defocusing the pulsed laser beam before applying the pulsed laser beam to the cartilage of the patient.

8. The method of claim 1, wherein the pulsed laser beam has a spot on the skin with a diameter between 4 and 10 millimeters.

9. The method of claim 1, wherein the pulsed laser beam has a spot on the skin with a diameter between 5 and 7 millimeters.

10. The method of claim 1, wherein the pulsed laser beam has an energy per pulse between 0.03 and 0.5 Joules.

11. The method of claim 1, wherein the pulsed laser beam has a pulse duration between 100 and 500 microseconds.

12. The method of claim 11, wherein the pulsed laser beam has a pulse duration between 100 and 300 microseconds.

13. The method of claim 1, wherein the pulsed laser beam has a pulse frequency between 5 and 100 Hertz.

14. The method of claim 1, wherein the pulsed laser beam has a pulse frequency between 5 and 50 Hertz.

15. A method of laser treatment for stimulating regeneration of cartilage in a patient, comprising:
generating a pulsed laser beam having kilowatts of peak power, a wavelength between 0.75 and 2.5 micrometers, and a pulse duration between 100 and 500 microseconds; and
applying the pulsed laser beam to the cartilage of the patient.

16. The method of claim 15, wherein the pulsed laser beam has a wavelength between 0.9 and 1.2 micrometers.

17. The method of claim 15, wherein the pulsed laser beam has a pulse frequency between 5 and 100 Hertz.

18. A method of laser treatment for treating tissue of a patient, the method comprising:
generating a pulsed laser beam, the energy of each pulse of the pulsed laser beam being above a threshold for cellular proliferation in tissue of the patient; and
applying the pulsed laser beam to the tissue at a duty cycle that allows heat accumulated during application of a single pulse of the pulsed laser beam to dissipate before application of a subsequent pulse.

19. A method as in claim 18 wherein the tissue is cartilage.

20. A method as in claim 18 wherein the pulsed laser beam is generated with a solid-state laser source.

21. A method as in claim 20 wherein the solid-state laser source is a Nd:YAG laser source.

22. A method as in claim 18 wherein the each pulse further has an average intensity sufficient to stimulate regeneration of the tissue.

23. A method as in claim 18 wherein the pulsed laser beam is applied to the tissue with a hand unit.

24. A method as in claim 18 wherein the pulsed laser beam is defocused before being applied to the tissue.

25. A method as in claim 18 further comprising controlling the temperature of the tissue by changing the duty cycle of the pulsed laser beam.
